(19) United States
(12) Patent Application Publication
(10) Pub. No.: US 2024/0240148 A1
(43) Pub. Date: Jul. 18, 2024

(54) ENGINEERING STEM CELLS FOR ALLOGENIC CAR T CELL THERAPIES

(71) Applicant: Appia Bio, Inc., Culver City, CA (US)

(72) Inventors: Jeenjoo S. Kang, Culver City, CA (US); Jeffrey Scott Wiezorek, Culver City, CA (US); Xi Wang, Culver City, CA (US); Michael Christopher, Culver City, CA (US)

(21) Appl. No.: 18/290,259

(22) PCT Filed: May 12, 2022
(86) PCT No.: PCT/US2022/028996

§ 371 Date: Nov. 10, 2023

Related U.S. Application Data
(60) Provisional application No. 63/188,872, filed on May 14, 2021.

(51) Int. Cl. C12N 5/0783; C12N 15/86

(52) U.S. Cl. CPC C12N 5/0646; C12N 15/86; C12N 2506/11; C12N 2510/00; C12N 2740/15043

(57) ABSTRACT

This disclosure provides methods for producing T cells with enhanced anti-tumor phenotypes. The T cells are made from hematopoietic stem cells by introducing into the hematopoietic stem cells a TCR, a CAR and at least one additional transgene.

### RELATED APPLICATION

The present application claims the benefit of and priority to U.S. provisional patent application Ser. No. 63/188,872, filed May 14, 2021, the content of which is incorporated by reference herein in its entirety.

### TECHNICAL FIELD

This disclosure relates to methods of engineering stem cells for use in allogenic cell therapies.

### BACKGROUND

The generation and expansion of T cells in vitro is useful for a broad range of clinical applications, including cancer treatment. For example, clinical data show engineered T cells with tumor antigen-specific receptors are useful in some patients to cause regression of metastatic cancer. Unfortunately, not all patients benefit from such treatment. One explanation is that during in vitro expansion, some T cells become exhausted or senescent, which limits therapeutic efficacy and persistence in vivo.

### SUMMARY

This disclosure provides systems and methods for producing T cells with enhanced anti-tumor phenotypes. In particular, this disclosure provides methods of making T cells from stem cells (e.g., HSCs) engineered with multiple transgenes including a T cell receptor (TCR), a chimeric antigen receptor (CAR), and at least one additional transgene. By starting with HSCs, systems and methods of the invention take advantage of self-regeneration and cellular differentiation capabilities of stem cells for the manufacture of T cells with improved anti-tumor phenotypes. In particular, this disclosure provides for introduction of nucleic acids, into CD34+ stem cells, that encode a TCR, a CAR, and at least one an additional transgene. The combined expression of TCRs and CARs is useful for providing T cells with specific cancer cell targeting properties. Moreover, endowed with at least one additional transgene, the T cells produced by methods of the invention are armed with cargo (e.g., cytokines) that, when in contact with the target cancer cell, is useful to treat the cancer.

Advantages of stem cells (e.g., HSCs) include their abilities for regeneration and expansion. Allogenic cell therapies often require billions of cells for a single dose of treatment. Due to a potentially limitless ability of stem cells for expansion, methods of the invention are well suited for producing high quality cellular products on a large scale and making those products rapidly available for treatment. Moreover, a hallmark of stem cells is their ability to differentiate into different cell types. In the context of this disclosure, the capacity for differentiation provides a cell manufacturing platform that may produce a broad array of T-cell subtypes, including, for example, natural killer T cells, alpha beta T cells, gamma delta T cells, among others.

In one aspect, this invention provides a method of producing an engineered invariant natural killer T cell. The method involves introducing into a hematopoietic stem cell (HSC) one or more nucleic acids encoding a T cell receptor (TCR), a chimeric antigen receptor (CAR), and at least one additional transgene; and transforming the HSC into an invariant natural killer T (iNKT) cell that expresses the TCR and CAR and comprises the transgene. In preferred embodiments, the one or more nucleic acids are provided by a single vector, such as, a lentiviral vector. However, in some instances, for example, when integrating large transgenes, it may be useful to integrate the one or more nucleic acids using at least two distinct nucleic acid molecules, such as, a plurality of vectors (e.g., lentiviral vectors). The at least two distinct nucleic acid molecules may be introduced into the HSCs sequentially or simultaneously.

In preferred embodiments, methods of the invention involve introducing nucleic acids into HSCs via lentiviral transduction. Introduction of the one or more nucleic acids provides for HSCs that express at least one TCR, CAR, and an additional transgene. Incorporation of the additional transgene is useful for providing therapeutic T cells with improved functional properties, such as, improved cell expansion, persistence, safety, and/or antitumor activities. The one or more additional transgenes may include any one or more of a cytokine, a checkpoint inhibitor, an inhibitor of transforming growth factor beta signaling, an inhibitor of cytokine release syndrome, or an inhibitor of neurotoxicity. For example, transgenes may be provided that encode one or more of IL-2, IL-7, IL-15, IL-12, IL-18, or IL-21.

Accordingly, in some instances, methods of the invention provide for the manufacture of CAR T cells with improved expansion and persistence capabilities by, for example, introducing nucleic acids encoding one or more of IL-2, IL-7, IL-1-15. In some instances, methods may provide CAR T cells with increased IFN-g production and thus improved T cell potency by, for example, introduction of transgenes encoding one or more of IL-12, IL-18. In other instances, methods of the invention are useful for enhancing naïve T cell production by introducing transgenes including IL-21. In some instances, methods described herein provide for the production of CAR T cells with improved safety properties by, for example, introducing inhibitors of L-6, GM-CSF, or other mediators of cytokine release syndrome and neurotoxicity. Methods may provide for CAR T cells with improved efficacy by providing payloads useful for combating tumor microenvironment, e.g., via inhibitors of TGF-B, checkpoints.

### BRIEF DESCRIPTION OF DRAWINGS

FIG. 1 diagrams a method for producing T cells.

FIG. 2 illustrates stages of ex vivo T cell manufacturing by three different processes.

FIG. 3 provides a comparison of two processes for producing T cells in vitro.

### DETAILED DESCRIPTION

Clinical studies of chimeric antigen receptor (CAR) T cells show remarkable results in treatment of certain pathologies, such as, B-cell malignancies. Presently, however, commercial methods involving CAR T cell therapy involve autologous CAR T cells whose widespread use is limited by logistics and high costs associated with ad hoc generation. Allogenic CAR T cell therapy address limitations of autologous cells by providing for pre-made cell stocks that are immediately available for patient treatment. Yet, despite its potential, methods for consistent production of therapeutically effective allogenic CAR T cells have not been established. Most protocols, for example, require long periods of ex vivo culture, with at least two activation steps, which potentially leads to over differentiation, T cell exhaustion, and/or cellular senescence, undermining in vivo efficacy. See, Jafarzadeh, 2020, Prolonged Persistence of Chimeric Antigen Receptor (CAR) T Cell in Adoptive Cancer Immunotherapy: Challenges and Ways Forward, Frontiers in Immunology, 11(702):1-17, incorporated by reference.

This disclosure provides reliable methods for manufacturing T cells with improved phenotype and cellular function. According to some aspects, this disclosure provides methods for T cell production with shortened ex vivo culture time. Certain methods of the invention reduce ex vivo culture by omitting ex vivo activation, which reduces ex vivo manufacturing processes by up to 2-3 weeks. Other methods of the invention provide for a single activation step. Methods of the invention recognize lengthy activation and/or expansion processes can occur in vivo, after administration to a subject. By shortening ex vivo cell culture, methods of the invention minimize opportunities for transcriptional and/or phenotypic changes to occur during T cell production. Furthermore, shortening ex vivo culture time reduces the amount of costly cell culture consumables that are needed for T cell maintenance.

In different aspect, this disclosure provides a multi-step workflow for making a T cell product with a single activation step. The method involves conducting a process comprising in vitro differentiation and maturation of an HSC into a T cell with no more than one in vitro T cell activation step. The T cell product can be used for a treatment or for research. Conventional methods of T cell manufacture require at least two separate in vitro T cell activation steps. Multiple activation steps generally involve multiple media types, i.e., media containing different activation factors. Advantageously, producing a T cell by a single activation step reduces time of cell culture and produces a more effective T cell product that expresses fewer markers associated with T cell exhaustion.

Methods of the invention are useful for producing T cell products with less in vitro cell culture. By reducing in vitro cell culture, methods of the invention produce T cell products that are more effective and contain fewer exhausted/dysfunctional T cells. The T cell products may express lower levels of proteins implicated in T cell exhaustion, e.g., PD-1, CTLA-4, LAG-3, TIM-3, 2B4/CD244/SLAMF4, CD160, TIGIT, than a T cell produced by 2 or more T cell activation steps. The T cell products may also express higher levels of IL-2.

Certain embodiments involve a multi-step process for producing a T cell wherein only one of the steps T cell activation. The single activation step can be performed by culturing the T cell in activation media, for example, media comprising T cell activation reagents such as antibodies.

In some instances, the activation step involves a peripheral blood mononuclear cell (PMBC) based activation. PMBC-based activation can involve introducing the T cell to alpha-galactosylceramide (aGC)-loaded PBMCs, soluble anti-CD3/28 positive PBMCs, and soluble anti-CD2/3/28 positive PBMCs. In some instances, the activation step involves an antigen presenting cell (aAPC) based T cell activation step. Accordingly, the activation step can involve introducing the T cell to aAPCs. Preferably, the aAPCs are irradiated. The aAPC may be an engineered K562 cell expressing CD80-CD83-CD137L-CAR-antigen. The aAPC may be an aAPC+CD1d, and/or aAPC+CD1d+/−aGC. In other instances, the activation step comprises a feeder free-based T cell activation step. The feeder free based T cell activation step can involve introducing, to the T cell, soluble antibodies including anti-CD3, anti-CD28, anti-CD2/3/28, CD3/28. In some embodiments, the method involves a culture media comprising one or more of IL-7/15, IL-2, IL-2+21, IL-12, or IL-18. In some embodiments, the media contains IL-15.

Moreover, according to other aspects, methods of the invention are useful to produce T cells with enhanced anti-tumor activities. This disclosure provides systems and methods for producing T cells from a stem cells (e.g., HSCs) incorporated with multiple transgenes including TCRs, CARs, and at least one additional transgene. By initiating a production process from stem cells, systems and methods of the invention take advantage of self-renewal and cellular differentiation capabilities for manufacture of T cells with “younger” phenotypes and enhanced anti-tumor activities. In particular, this disclosure provides for introduction of nucleic acids, into CD34 positive stem cells, that encode for at least one TCR, CAR, and at least one an additional transgene. The combined expression of TCRs and CARs and the additional transgene is useful for providing T cells with specific cancer cell targeting properties useful to treat the cancer.

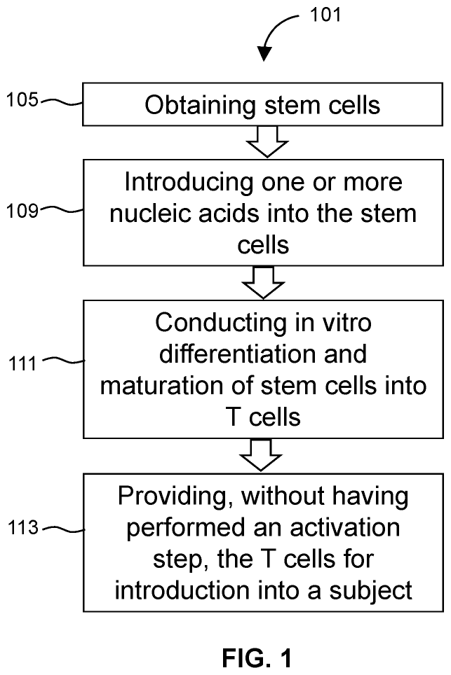

FIG. 1 diagrams a method 101 for producing T cells. In particular, illustrated is a simple flow diagram to provide a general overview of methods for producing T cells according to aspects of the invention. The method 101 includes obtaining 105 stem cells (e.g., CD34+ hematopoietic stem/progenitor cells); introducing 109 into the stem cells one or more nucleic acids (e.g., encoding TCRs, CARs, and additional transgenes); conducting 111 an in vitro differentiation and maturation of the stem cells to produce T cells; and providing 113 (e.g., for allogenic therapy or research) the cells without having performed a T cell activation step.

The method 101 involves obtaining 105 stem cells. Preferably, the cells are CD34+ cells. In one non-limiting example the CD34+ stem cells are a hemopoietic stem/progenitor cells. Hematopoietic stem or progenitor cells are stem cells that give rise to other blood cells in a process referred to as haematopoiesis.

The hematopoietic stem/progenitor cells may be obtained from a healthy donor. The hematopoietic stem/progenitor cells may be obtained from, for example, bone marrow, peripheral blood, amniotic fluid, or umbilical cord blood. The hematopoietic stem/progenitor cell may be obtained from umbilical cord blood by clamping ends of an umbilical cord and aspirating blood from between the clamped ends with a needle. The hematopoietic stem/progenitor cells may be isolated from cord blood using positive immunomagnetic separation techniques, and citrate-phosphate-dextrose (CPD) may be added to the cord blood as an anticoagulant. The cells from the cord blood may be cryopreserved and stored at a temperature of, for example, −80 degrees Celsius until use.

In practicing methods of the disclosure, obtaining 105 the stem cells preferably involves receiving a vial of cryopreserved CD34+ cord blood cells including hemopoietic stem/progenitor cells. The vial of cryopreserved cord blood cells may be received from a cell bank in an insulated container on dry ice, for example.

The vial of cryopreserved cord blood cells may be thawed according to methods known in the art. For example, the vial of cells may be thawed by placing the vial into a 37-degree water bath for approximately 1 to 2 minutes. In some preferred embodiments, once the cells are thawed, the cells are plated onto tissue culture dishes pre-coated with a reagent that promotes colocalization of a virus with target cells to enhance transduction efficiency.

The CD34+ cells may be plated in a standard 6 well dish at, for example, 10,000 cells per well, 15,000 cells per well, or 20,000 cells per well, or 25,000 cells per well, or more. Preferably, the cells are plated at 15,000 cells per well.

The method 101 further includes introducing 109, into the CD34+ stem cells, one or more nucleic acids encoding for one or more of a TCR, a CAR, and/or an additional transgene. Preferably, the one or more nucleic acids encode at least two of a TCR, a CAR, or an additional transgene. For example, in some embodiments, the one or more nucleic acids introduced 109 into stem cells encode at least a TCR and a CAR, to produce a T cells capable of targeting a specific protein expressed on a surface of cancer cells. In other embodiments, the one or more nucleic acids introduced 109 into the stem cells encode for each of a TCR, a CAR, and an additional transgene.

In preferred embodiments, the TCR introduced by way of nucleic acid is an iNKT TCR. The iNKT TCR may include one of an alpha chain of an iNKT cell receptor, a beta chain of an iNKT cell receptor, or both. Preferably, the iNKT cell receptor is expressed by the stem cells such that the stem cells recognizes alpha-Galactosylceramide. In addition, preferred embodiments include introducing nucleic acids encoding at least one CAR. The CAR, as discussed below, may be of a first generation, a second generation, or a third generation CAR. The CAR is useful to provide cells with a receptor specific to an antigen associated with cancer. For example, in some embodiments the antigen comprises one of Mesothelin, Glypican 3, CD19, or BCMA.

T cells produced by methods of the invention may be genetically modified to express at least one additional transgene. The transgene may be, for example, one of a cytokine, a checkpoint inhibitor, an inhibitor of transforming growth factor beta signaling, an inhibitor of cytokine release syndrome, or an inhibitor of neurotoxicity. Accordingly, methods of the invention may be useful to produce T cell with enhanced effector function.

For example, in some instances, methods of the invention are useful for the manufacture of CAR T cells with improved expansion and persistence capabilities, which is provided by introduction of transgenes encoding one or more of IL-2, IL-7, IL-1-15. In some instances, methods may provide CAR T cells with increased IFN-g production and thus improved T cell potency by, for example, introduction of transgenes encoding one or more of IL-12, IL-18. In some instances, methods of the invention are useful for enhancing naïve T cell production by introducing transgenes including IL-21. In some instances, methods described herein provide for the production of CAR T cells with improved safety properties by, for example, introducing inhibitors of IL-6, GM-CSF, or other mediators of cytokine release syndrome and neurotoxicity. Methods may provide for CAR T cells with improved efficacy by providing payloads useful for combating tumor microenvironment, e.g., via inhibitors of TGF-B, checkpoints.

Introducing 109 the one or more nucleic acids into the stem cells may be accomplished by viral transduction method or a non-viral transfection. In some instances, for example, methods for introducing the one or more nucleic acids involve non-viral methods, for example, using a Sleeping Beauty transposon/transposase system. The Sleeping Beauty transposon system involves a synthetic DNA transposon designed to introduce precisely defined DNA sequences into the chromosomes of cells. The system uses a Tc1/mariner-type system, with the transposase resurrected from multiple inactive fish sequences. Advantageously, non-vial methods may provide for cost-savings benefit and reduce risks associated with use of certain virus. However, non-viral methods may be associated with reduced efficiency. As such, preferred embodiments introduce nucleic acids into stem cells by viral transduction, e.g., via a retrovirus.

Viral transduction methods are well recognized for their versatility and involve the use of lentiviral vectors, which are useful to transduce both dividing and nondividing cells with significant amounts of nucleic acid. The use of lentiviral vectors is considered safe and often provides long-term transgene expression. Accordingly, the method 101 preferably introduces 109 the one or more nucleic acids into the 34+ stem cells via a lentiviral transduction. For discussion on lentiviral transduction of stem cells, see Jang, 2020, Optimizing lentiviral vector transduction of hematopoietic stem cells for gene therapy, Gene Therapy (27): 545-556, which is incorporated by reference.

Methods of the invention are not limited by any one process or laboratory procedure for introducing nucleic acids into stem cells. In some instances, a single lentiviral vector is used. The single lentiviral vector may encode each of a TCR, a CAR, and an additional transgene. In other instances, at least two distinct lentiviral vectors are used, wherein each one of the at least two lentiviral vectors encode at least one of a TCR, a CAR, and an additional transgene, such that, upon transduction, each of the TCR, the CAR, and the additional transgene are transduced into the stem cells. Moreover, in instances wherein more than one lentiviral vector is used, the method 101 is not limited by the temporal sequence of introducing the two (or more) lentiviral vectors the stem cells. The vectors may be introduced concurrently, in the same transduction, or sequentially.

The method 101 further involves conducting 111 a process comprising in vitro differentiation and maturation of the stem cell (e.g., HSC) into a T cell.

One advantage of the cell manufacturing method 101 lies in the ability to produce a broad array of T cell subtypes from a single starting material, i.e., stem cells. T cells produced by methods of the invention may include, for example, helper T cells, cytotoxic T cells, memory T cells, regulatory T cells, natural killer T cells, invariant natural killer T cells, alpha beta T cells, gamma delta T cells. In preferred embodiments, the method 101 involves producing invariant natural killer T cells. Production of invariant natural killer T (iNKT) cells are preferred for their allogenic cell therapy applications. In particular, its ability to activate and expand antigen-specific T cell responses to treat cancer without inducing graft versus host disease.

Accordingly, the method 101 includes conducting 111 an in vitro differentiation and maturation process of stem cells (e.g., HSCs) into T cells (e.g., iNKT). As discussed in detail below, conducting 111 in vitro differentiation and maturation of the stem cells is preferably done with the proviso that the process does not involve subsequent in vitro steps of activation and expansion of the T cell.

Differentiation of CD34 positive cells into T cells may occur in stages. A first stage may involve in vitro differentiation of CD34 positive stem cells into CD4 and CD8 double negative T cells. Differentiation of the CD34 positive stem cells generally involves introducing CD34 positive stem cells to a combination of cytokines and/or chemokines in culture, e.g., 1-2 weeks. In some instances, the cytokines and/or chemokines may be provided by commercially available progenitor expansion supplements, such as, the supplement sold under the trade name StemSpan by STEMCELL. Embodiments of conducting 111 the in vitro process further involve maturation of CD4 and CD8 double negative T cells into CD 4 and CD 8 double positive cells. In some instances, maturating the double negative cells involves culturing the cells in a commercially available progenitor maturation medium, such as, the progenitor maturation medium provided under the trade name StemSpan by STEMCELL. In some embodiments, the cells are be cultured in progenitor maturation medium for 7 days.

According to certain embodiments of the method 101, conducting 111 the in vitro differentiation and maturation process of CD34+ stem cells into T cells produces CD4 positive CD8 positive T cells. The CD4 positive CD8 positive T cells may be naïve T cells. Naive T cells are commonly characterized by surface expression of L-selectin (CD62L) and C-C Chemokine receptor type 7 (CCR7). In some instances, the absence of the activation markers CD25, CD44 or CD69, and the absence of memory CD45RO isoform. Naïve T cells may also express functional IL-7 receptors, consisting of subunits IL-7 receptor-alpha, CD127, and common-gamma chain, CD132. The naïve T cells may be cryopreserved for storage or introduced into a subject for during an allogenic cell therapy treatment. Inside the subject, the naive T cells may circulate through peripheral lymphatics awaiting initial antigenic stimulation. Upon initial stimulation through the naïve cells' TCRs, the cells begin to modulate expression of surface molecules associated with activation, co-stimulation, and adhesion. The expression pattern of these molecules may be used to further define effector and antigen-experienced of T cell subsets.

In some embodiments, the CD4 positive CD8 positive T cells are expanded in vitro prior to cryopreservation and/or administration to an allogenic cell therapy recipient. Expansion of the CD4 positive CD8 positive T cells may involve culturing the cells in the presence of on or more of IL-7, IL-15, CD3, CD28, CD2, alpha-galactosylceramide.

Methods of the invention take advantage of in vivo activation mechanisms to reduce in vitro culture steps. Once a T cell has been produced, without having undergone two activations steps, and is introduced into a subject's body, the T cell is fully activated when the T cell encounters a properly activated antigen presenting cell (APC), such as a dendritic cell, for example, at secondary lymphoid organ. If the APC displays an appropriate peptide ligand through the major histocompatibility complex (MHC) class II molecule, it is recognized by the TCR. This is important for activating the T cell. Two other stimulatory signals delivered by the APC may also be required. These signals can be provided by two different ligands on the APC surface, such as CD80 and CD86, to a surface molecule on the T cell, e.g., CD28. Other factors important for activation include those factors involved in directing T cell differentiation into different subsets of effector T cells, e.g., cytokines, such as IL-6, IL-12 and TGF-β. The CD28-dependent co-stimulation of activated T cells can lead to production of IL-2 by the activated T cell themselves. Following expression of IL-2, there can also be an upregulation of the third component (called α-chain) of the IL-2 receptor, also known as CD25, in addition to other regulatory molecules such as ICOS and CD40L. Binding of IL-2 to its high affinity receptor promotes cell growth, whilst APCs, mainly dendritic cells generate various cytokines or express surface proteins that induce the differentiation of CD4+T lymphocytes into cytokine producing effector cells, depending on environmental conditions.

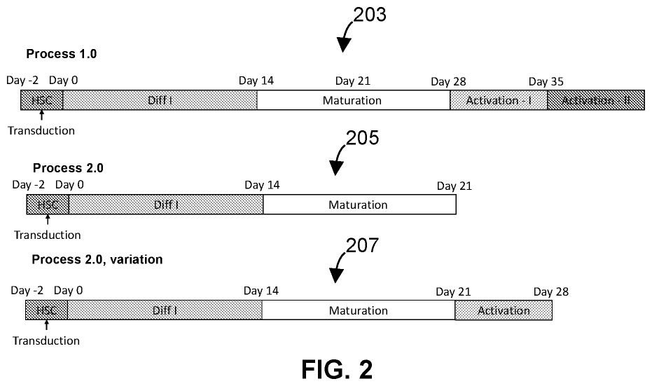

FIG. 2 illustrates stages of ex vivo T cell manufacturing by three different processes. Specifically, illustrates a conventional ex vivo process 203 for making T cells in comparison with reduced ex vivo processes 205, 207 of the invention. In the conventional process 203, matured T cells are subjected to at least two rounds of in vitro activation steps, two of which are illustrated. This process 203 can requires at least 35 days of ex vivo cell culture complete, e.g., at least 42 days, and often times longer. Conversely, process 205 omits T cell activation thus can generate T cells within as few as 21 days. By omitting the activation step, ex vivo culture of T cells is substantially reduced, e.g., 2-3 weeks. A third process 207, involves a single activation step. The single activation step may be helpful for producing effective quantities of T cells. By using only one activation—and not two activation steps—methods of the invention can generate T cells in at least 14 days less than prior art T cell manufacturing processes.

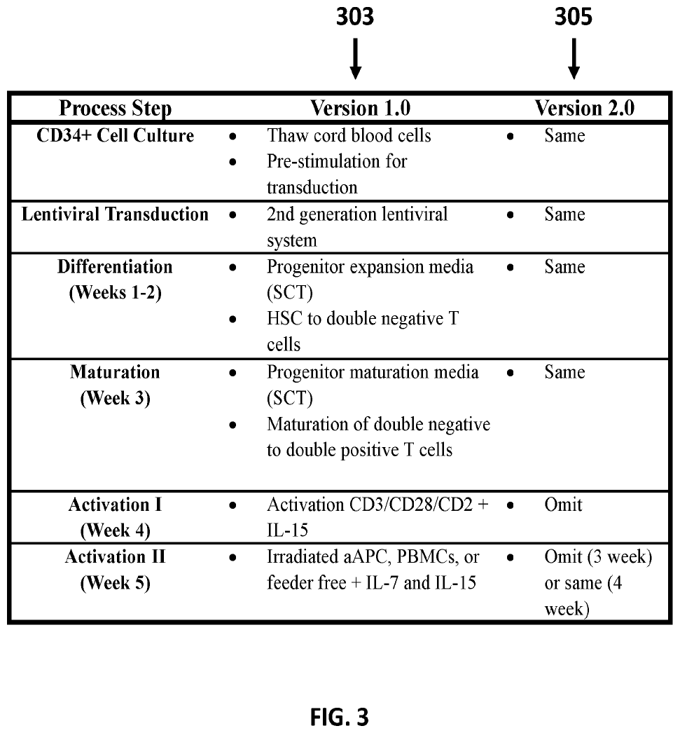

FIG. 3 provides a comparison of two culture processes for producing T cells in vitro. A first process 303 includes two activation steps. A two-step activation process, which is accomplished by treating double positive T cells with, for example, different activation media comprising CD3/CD28/CD2 and IL-15, increases manufacturing process by at least one week and generally more. A second process 305, omits in vitro activation.

Methods of the invention may involve a single activation step. A single activation step can involve culturing T cells with activation reagents for a period of time no longer than 7 days. In other embodiments, a single activation step involves culturing a T cell in activation media for no longer than 6 days or 5 days or 4 days or 3 days or 2 days or 1 day. In other embodiments, a single activation step comprises not culturing a T cell in activation media for longer than 8 days, or 9 days, or 10 days or 11 days, or 12 days or 13 days or 14 days. A single activation step can involve culture T cells with a single type of activation media. The single type of activation media can include activation reagents, such as soluble antibodies. The single activation step can involve co-culturing the T cells with antigen presenting cells, such as aAPCs. The single activation step can involve co-culturing the T cells with PBMCs.

Methods of the invention involve production of T cells from hemopoietic stem/progenitor cells. The hemopoietic stem/progenitor cells generally related to CD34+ cells that may be found in cord blood. In some instances, the cells may be derived from a progenitor cell. In some instances, the cell is a pluripotent stem cell, such as, an embryonic stem cell.

Allogeneic CAR T cells produced from HSCs may provide a curative therapeutic approach for certain pathologies. However, some limitations include GVHD, a donor T-cell-mediated alloreactive process responsible for much of the morbidity and mortality associated with allogenic cell therapies. Some clinical research show that donor iNKT cells can prevent GVHD without increasing the risk of disease relapse. Adoptive transfer of donor CAR iNKT cells followed by in vivo activation and/or expansion, may prevent or alleviate symptoms of GVHD. This protective effect may be mediated through Th2 polarization of alloreactive T-cells and expansion of donor regulatory T-cells (Tregs). Since allogeneic iNKT-cells, as produced by methods of the invention, do not cause GVHD, methods described herein provide an ideal platform for ‘off-the-shelf’ CAR immunotherapy.

Methods of the invention are useful to manufacture therapeutically active T cells that acquire antigen-specificity via functional rearrangements of antigen recognition regions of TCRs. The TCR is a molecule found on the surface of T cells (or T lymphocytes) that is responsible for recognizing antigens bound to major histocompatibility complex molecules. The TCR may be composed of at least two different protein chains (e.g., a heterodimer). In most (e.g., 95%) T cells, this consists of an alpha and beta chain, whereas in some (e.g., 5%) T cells, this consists of gamma and delta chains. Such T cells may have antigen-specificity in cell surface TCR molecules differentiate in vivo into different phenotypic subsets, including, but not limited to, classical CD3 positive, alpha-beta TCR CD4 positive, CD3 negative alpha-beta TCR CD8 positive, gamma delta T cells, Natural Killer T cells, etc. Furthermore, T cell may further include various activation states, including, but not limited to, naive, central memory, effector memory, terminal effector, etc.

In preferred embodiments, methods of the invention provide for the manufacture of CAR T cells. CAR T cells are T cells that have been genetically engineered to produce an artificial T-cell receptor for use in immunotherapy. CARs (i.e., chimeric antigen receptors) can be used to graft the specificity of a monoclonal antibody onto stem cells with TCRs via transfer of their coding sequences facilitated by, for example, retroviral vectors. The CARs are receptor proteins that have been engineered to give T cells the new ability to target a specific protein. The receptors are chimeric because they combine both antigen-binding and T-cell activating functions into a single receptor. Accordingly, methods of the invention provide products for immunotherapy by producing modified T cells that recognize cancer cells in order to more effectively target and destroy them.

In practicing methods of the invention, any CAR suitable for engineering effector cells (e.g., T cells) as used in adoptive immunotherapy therapy, may be used in the present invention. CARs that can be used in the present invention include those described in Kim and Cho, 2020, Recent Advances in Allogeneic CAR-T Cells, Biomolecules, 10(2):263, which is incorporated by reference.

CARs generally include an extracellular domain, a transmembrane domain and an intracellular domain. The extracellular domain may include an antigen binding/recognition region/domain. The antigen binding domain of the CAR is useful to bind to a specific antigen, e.g., a tumor antigen, a pathogen antigen (e.g., viral antigen), a CD (cluster of differentiation) antigen. The extracellular domain may also include a signal peptide that directs nascent protein into the endoplasmic reticulum. Signal peptide may be essential if the CAR is to be glycosylated and anchored in the cell membrane. The transmembrane domain is a hydrophobic alpha helix that spans the membrane. Different transmembrane domains result in different receptor stability. After antigen recognition, receptors cluster and a signal is transmitted to the cell. The most commonly used intracellular component is CD3Xi which contains 3 ITAMs. This transmits an activation signal to the T cell after antigen is bound. CARs can also include a spacer region that links the antigen binding domain to the transmembrane domain. The spacer region should be flexible enough to allow the antigen binding domain to orient in different directions to facilitate antigen recognition. The spacer can be the hinge region from IgG1, or the CH2CH3 region of immunoglobulin and portions of CD3.

Presently, there are three generations of CARs. First generation CARs typically comprise an antibody derived antigen recognition domain (e.g., a single-chain variable fragments (scFv)) fused to a transmembrane domain, fused to cytoplasmic signaling domain of the T cell receptor chain. First generation CARs typically have the intracellular domain from the CD3 Xi-chain, which is the primary transmitter of signals from endogenous TCRs. First generation CARs can provide de novo antigen recognition and cause activation of both CD4+ and CD8+ T cells through their CD3 Xi chain signaling domain in a single fusion molecule, independent of HLA-mediated antigen presentation. In one non-limiting example, T cells can be genetically engineered to express artificial TCRs that direct cytotoxicity toward tumor cells, for discussion, see Eshhar 1993, Specific activation and targeting of cytotoxic lymphocytes through chimeric single chains consisting of antibody-binding domains and the gamma or zeta subunits of the immunoglobulin and T-cell receptors, Proc Natl Acad Sci, 90, 720-724, incorporated by reference. Second generation CARs are similar to first generation CARs but include two co-stimulatory domains, such as, CD28 or 4-1BB. The involvement of these intracellular signaling domains improve T cell proliferation, cytokine secretion, resistance to apoptosis, and in vivo persistence. Third generation CARs combine multiple co-stimulatory domains, such as CD28-41BB or CD28-OX40, to further augment T cell activity.

In some instances, methods of the invention involve performing genetic modification of HSCs to provide for an additional transgene (i.e., a transgene in addition to one of a CAR or TCR). For example, in some embodiments, the additional transgene encodes one or more cytokines. Cytokines relate to substances, such as interferon, interleukin, and growth factors, which are secreted by certain cells of the immune system and influence other cells. According to embodiments of the invention, transgenes encoding one or more of IL-2, IL-7, IL-15, IL-12, IL-18, or IL-21, may be provided to facilitate T cell function or tumor efficacy.

Introduction of one or more transgenes into CAR T cells may improve properties such as T cell expansion and persistence, (e.g., using IL-2, IL-7/15), IFN-g production and T-cell potency (e.g., with IL-12, IL-18), enhancing naïve subsets (e.g., IL-21), improve safety (e.g., via inhibitors of IL-6, GM-CSF or other mediators of CRS and neurotoxicity), or improve efficacy by combating the tumor microenvironment (TGF-B, checkpoints, etc.).

For example, IL-12 and IL-18 play a major role in augmenting certain effector functions of CAR T cells. IL-12 is known to activate certain NK cells and T lymphocytes, induce Th-1 type responses, and increase IFN-gamma secretion. The inducible expression of IL-12 may augment antitumor capabilities of CAR T cells against certain pathologies, such as, lymphoma, hepatocellular carcinoma, ovarian tumors, and B16 melanoma. IL-18 has also been used to improve the therapeutic potential of CAR T cells. Initially identified as a potent inducer of IFN-gamma, IL-18 may contribute to T and NK cell activation and Th-1 cell polarization. For example, Meso-targeted CAR T cell may be provided with transgenes encoding IL-18 to augment the secretion of IFN-gamma and to eradicate cancer cells. For further discussion, see, Tian, 2020, Gene modification strategies for next-generation CAR T cells against solid cancers, Journal of Hematology & Oncology, volume 13(5), incorporated by reference.

IL-7, IL-15, and IL-21 are useful for promoting generation of stem cell-like memory T cell phenotype. This phenotype may provide for increased expansion and persistence of T cells in vivo. In some instances, transgenes encoding IL-2 may be provided. Producing T cells with IL-2 may provide T cells with improved capacities for responding to tumor environments by, for example, facilitating induction and the production of proteins involved in nutrient sensing and uptake.

In some embodiments, the transgene is an inhibitor of cytokine release syndrome. Cytokine release syndrome relates to a serious, potentially life-threatening side effect often associated with CAR T-cell therapy. Cytokine release syndrome manifests as a rapid (hyper)immune reaction driven by excessive inflammatory cytokine release, including, for example, IFN-gamma and IL-6. Many cytokines implicated in cytokine release syndrome are known to operate through a JAK-STAT pathway. Accordingly, in some embodiments, methods of the invention involve producing CAR-T cells that express inhibitors of the JAK pathway to improve in vivo CAR T-cell proliferation, antitumor activity, and cytokine levels. For example, transgenes may be provided that inhibit function of IL-6, JAK-STAT, or BTK. Moreover, the inhibitors may further be useful for inhibition of neurotoxicity. CAR T cell related neurotoxicity is a syndrome that often leads to severe neurologic disturbances such as seizures and coma.

In other instances, methods involve introducing a transgene into HSCs, wherein the transgene is a checkpoint inhibitor, e.g., an immune checkpoint inhibitor. Immune checkpoints are regulators of certain aspects of immune systems. In normal physiological conditions, checkpoints enable the immune system to respond to host antigens preserving healthy tissues. In cancer, these molecules facilitate tumor cell evasion. In some instances, transgenes may encode antibodies or antibody fragments, such as, anti-cathepsin antibodies, galectin-1 blockade and anti-OX40 agonistic antibodies. The antibodies may be secreted or expressed on surfaces of cells. The antibodies may be secreted that, for example, target PD1 or PDL1.

In embodiments, CAR T cells expressing an inhibitor of transforming growth factor beta are produced. Engineered cells face hostile microenvironments which limit their efficacy. Modulating the environments may convert be useful for facilitating CAR T cells ability to proliferate, survive and/or kill cancer cells. One of the main inhibitory mechanisms within the tumor environment is transforming growth factor beta. Accordingly, some aspects of the invention involve introducing transgenes encoding inhibitors of transforming growth factor beta. The inhibitors may be, for example, antibodies or fragments thereof. The antibody or antibody fragments may be secreted from CAR T cells to interfere with normal functions of transforming growth factor beta.

In some embodiments, methods of include making CAR T cells to target solid tumor types through markers of tumor microenvironment. In other embodiments, methods make CAR T cells with a single-domain antibody (VHH)-based chimeric antigen receptor, which can be used to recognize markers of a tumor microenvironment without the need for tumor-specific targets. VHH-based CAR T cells, according to the invention, may target the tumor microenvironment through immune checkpoint receptors or through stroma and extra cellular matrix markers, which effective against solid tumors in syngeneic, immunocompetent animal models. Accordingly, methods of the invention are useful to make CAR T cells that target tumors which may lack tumor-specific antigen expression. The variable regions of heavy-chain-only antibodies (VHHs or nanobodies) are small, stable, camelid-derived single-domain antibody fragments with affinities comparable to traditional short chain variable fragments (scFvs). VHHs are generally less immunogenic than scFvs and, owing to their small size, can access epitopes different from those seen by scFvs. VHHs, as provided by the invention, can therefore serve as suitable antigen recognition domains in CAR T cells. Unlike scFvs, VHHs do not require the additional folding and assembly steps that come with V-region pairing. They allow surface display without the requirement for extensive linker optimization or other types of reformatting. The ability to switch out various VHH-based recognition domains yields a highly modular platform, accessible without having to reformat each new conventional antibody into an scFv.

Moreover, many microenvironments involve expression of inhibitory molecules such as PD-L1. Using VHHs as recognition domains, e.g., PD-L1-specific CAR T cells, CAR T cells produced by methods of the invention can target the tumor microenvironment. PD-L1 is widely expressed on tumor cells, as well as on the infiltrating myeloid cells and lymphocytes. A CAR that recognizes PD-L1 should relieve immune inhibition and at the same time allow CAR T cell activation in the tumor microenvironment. PD-L1-targeted CAR T cells might thus reprogram the tumor microenvironment, dampening immunosuppressive signals and promoting inflammation. For example, as discussed in Xie, 2019, Nanobody-based CAR T cells that target the tumor microenvironment inhibit the growth of solid tumors in immunocompetent mice, PNAS Apr. 16, 2019 116 (16) 7624-7631, which is incorporated by reference.

According to embodiments of the present disclosure, CD34+ stem cells are genetically engineered to express one or more of a CAR, a TCR, and an additional transgene (e.g., a cytokine). For initial genetic modification of the cells to provide for tumor or viral antigen-specific cells, a retroviral vector may be used for viral transduction. Combinations of retroviral vector and an appropriate packaging infecting human cells in culture are known in the art. In preferred embodiments, a third-generation lentiviral vector may be used. The vector may be modified with cDNA sequences containing sequences of antibodies or antibody fragments to target preferred antigens. For example, as described in Carpenito, 2008, Control of large, established tumor xenografts with genetically retargeted human T cells containing CD28 and CD137 domains, PNAS, 106(9) 3360-3365; Li, 2017, Redirecting T Cells to Glypican-3 with 4-1BB Zeta Chimeric Antigen Receptors Results in Th1 Polarization and Potent Antitumor Activity, Human Gene Therapy, 28(5): 437-448; Adusumilli, 2014, Regional delivery of mesothelin-targeted CAR T cell therapy generates potent and long-lasting CD4-dependent tumor immunity, Science Translational Medicine, 261(6): 1-14; each of which are incorporated by reference.

Some aspects of the disclosure involve introducing and expressing multiple transgenes in HSCs. To facilitate the expression of multiple genes, it may be useful to separate the transgenes, on nucleic acids, with 2A sequences, i.e., coding domains of 2A peptides. 2A self-cleaving peptides, or 2A peptides, is a class of 18-22 aa-long peptides, which can induce ribosomal skipping during translation of a protein. Inside the cell, when the coding domains of a 2A peptide is inserted between two coding domains of two proteins (e.g., TCR and CAR), the peptide will be translated into two proteins folding independently due to ribosome skipping.

Methods of the invention are useful to transform engineered HSCs into T cells for clinical application. Methods of transformation generally include differentiation of HSCs into T cells. Cellular differentiation is the process in which a cell changes from one cell type to another. Usually, the cell changes to a more specialized type. Differentiation of HSCs into T cells may involve multiple stages of differentiation. As a first stage, CD34+ cells may be differentiated into CD4 CD8 double negative T cells. Generation of double negative T cells can be achieved by culture of CD34+ cells in the presence of a cocktail of cell factors including hematopoietic cytokines. The cocktail may include SCF (e.g., hSCF), Flt3L (e.g., hFlt3L), and at least one cytokine, and bFGF for hematopoietic specification. The cytokine can be a Th1 cytokine, which includes, but is not limited to IL-3, IL-15, IL-7, IL-12 and IL-21. The cells may be immunophenotypically analyzed by FACS for expression of CD34, CD31, CD43, CD45, CD41a, ckit, Notch1, IL7Rα.

Double negative T cells may be further differentiated via an antigen-independent maturation process to produce functional, inactivated, T cells. This process may involve culturing double negative T cells in a lymphoid progenitor expansion medium. The media may include, for example, a feeder cell and SCF, Flt3L and at least one cytokine. The cytokine may be a Th1 cytokine, which includes, but is not limited to, IL-3, IL-15, IL-7, IL-12 and IL-21. In some embodiments, the cytokine may enhance survival and/or functional potential of the cells.

Cell products comprising T cells, including T cells that have not undergone an activation and/or expansion step, can be provided systemically or directly to a subject for the treatment of a neoplasia, pathogen infection, or infectious disease. In one embodiment, T cells of the present invention may be directly injected into an organ of interest (e.g., an organ affected by a neoplasia). Alternatively, T cells and compositions comprising thereof can be provided indirectly to the organ of interest, for example, by administration into the circulatory system (e.g., the tumor vasculature). Preferably, activation and expansion of the T cells occurs in vivo, after introduction into a subject.

T cells and compositions comprising thereof of the present invention may be administered in any physiologically acceptable vehicle, normally intravascularly, although they may also be introduced into bone or other convenient site where the cells may find an appropriate site for regeneration and differentiation (e.g., thymus). Usually, at least 100,000 cells will be administered, and sometimes 10,000,000,000 cells, or more.

Methods of the invention provide for compositions of cells that may be combined with pharmaceutical compositions for administration of an allogenic cell therapy. When administering a therapeutic composition of the present invention (e.g., a pharmaceutical composition comprising CAR T cells derived from HSCs), it will generally be formulated in a unit dosage injectable form (solution, suspension, emulsion). The compositions may be provided in a therapeutically effective concentration. The therapeutically effective concentration is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the antigen-binding fragment administered.

For adoptive immunotherapy using antigen-specific T cells of the invention, cell doses in the range of 10,000,000-10,000,000,000 may be infused. Upon administration of the T cells into the subject T cells may undergo an antigen-dependent activation process.

This disclosure provides methods for manufacture of T cells for cell therapies and/or research. In some aspects, methods provide economical methods of T cell manufacture by reducing time of ex vivo cell culture. In some related aspects, methods of the invention provide for the manufacture of T cells with enhanced cytotoxic efficacy. Prolonged cell culture has previously been associated with transcriptional and phenotypic changes of certain cell types. Although transcriptional and phenotypic changes of T cells in culture are poorly characterized, this disclosure recognizes that unintended changes of cells during prolonged culture may account for observed reductions in therapeutic efficacy and product batch variability identified in allogenic cells. For example, prolonged cell culture of T cells may give rise to elevated levels of exhaustion markers, which reflect loss of effector function. For example, prolonged culture may be associated with increased expression of PD1, LAG3. CD244, CD160, for further discussion, see Wherry, 2016, Molecular and cellular insights into T cell exhaustion, Nat Rev Immunol, 15(8): 486-499, which is incorporated by reference. By shortening ex vivo manufacture, methods of the invention are useful for consistent production of therapeutically effective T cells.

Accordingly, in one aspect, this disclosure provides a method of producing a T cell. The method involves conducting a process involving in vitro differentiation and maturation of a hematopoietic stem cell (HSC) into a T cell, with the proviso that the process does not involve subsequent in vitro steps of activation and/or expansion of the T cell. Rather, activation and/or expansion of the T cell preferably occurs in vivo after introduction into the subject. Advantageously, omitting in vitro activation and/or T cell expansion saves weeks (e.g., at least two weeks) off conventional T cell manufacturing processes, which may be useful for producing a T cell with enhanced cytotoxic efficacy.

Methods of the invention are useful for producing allogenic therapies that are safe and effective. In some instances, methods may involve characterizing cell products at one or more points during manufacture to ensure product quality. In some embodiments, methods of the invention involve analyzing T cells to identify one or more proteins expressed by the T cells. The one or more proteins may include one or more CCR7, CD62L, or CD45RA. The proteins may include markers associated with naïve stem cells. Analyzing preferably includes high throughput methods of analyzing cell surface proteins, e.g., methods based on fluorescent signals of individual cells in bulk, such as, FACS.

On demand availability of treatment is one benefit of allogenic cell therapies. Since methods may involve manufacture of cells before clinical application, some preferred methods may include cryopreserving T cells. Cryopreserving T cells is useful for safe and effective storage of cells until they are needed by a patient. Cryopreserving is also useful for transportation of cell products to clinical facilities where they can be administered to patients. In some preferred embodiments, double positive T cells (e.g., naïve T cells) are cryopreserved without preforming an in vitro activation step.

Over the past decade, immunotherapy has become the new-generation cancer medicine. In particular, cell-based cellular therapies have shown great promise. An outstanding example is the CAR-engineered adoptive T cells therapy, which targets certain blood cancers at impressive efficacy. However, most of the current protocols for treatment consist of autologous adoptive cell transfer, wherein immune cells collected from a patient are manufactured and used to treat this single patient. Such an approach is costly, manufacture labor intensive, and difficult to broadly deliver to all patients in need. Allogenic immune cellular products, by methods described herein, can be manufactured at a large-scale and can be readily distributed to treat a higher number of patients therefore are in great demand.

Some embodiments concern an engineered iNKT cell or a population of engineered iNKT cells. In at least some cases, the engineered iNKT cells comprise CAR and/or engineered T cell receptor. Any embodiment discussed in the context of a cell can be applied to a population of such cells. In particular embodiments, an engineered iNKT cell comprises a nucleic acid comprising 1, 2, and/or 3 of the following: i) all or part of an invariant alpha T-cell receptor coding sequence; ii) all or part of an invariant beta T-cell receptor coding sequence, or iii) a suicide gene. In further embodiments, there is an engineered iNKT cell comprising a nucleic acid having a sequence encoding: i) all or part of an invariant alpha T-cell receptor; ii) all or part of an invariant beta T-cell receptor, and/or iii) a suicide gene product.

Further aspects relate to engineered iNKT cells with increased levels of NK activation receptors, decreased levels of NK inhibitory receptors, and/or increased levels of cytotoxic molecules. In some embodiments, the NK activation receptors comprise NKG2D and/or DNAM-1. In some embodiments, cytotoxic molecules comprise Perforin and/or Granzyme B. In some embodiments, the inhibitor receptors comprise KIR. The increase or decrease may be with respect to the levels of the same marker in non-engineered iNKTs isolated from a healthy individual. Further aspects relate to a population of engineered iNKT cells, wherein the population of cells has increased levels of NK activation receptors, decreased levels of NK inhibitory receptors, and/or increased levels of cytotoxic molecules.

In some embodiments, the engineered iNKT cell comprises a nucleic acid under the control of a heterologous promoter, which means the promoter is not the same genomic promoter that controls the transcription of the nucleic acid. It is contemplated that the engineered iNKT cell comprises an exogenous nucleic acid comprising one or more coding sequences, some or all of which are under the control of a heterologous promoter in many embodiments described herein.

In a particular embodiment, there is an engineered invariant natural killer T (iNKT) cell that expresses at least one invariant natural killer T-cell receptor (iNKT TCR) and an exogenous suicide gene product, wherein the at least one iNKT TCR is expressed from an exogenous nucleic acid and/or from an endogenous invariant TCR gene that is under the transcriptional control of a recombinantly modified promoter region. An iNKT TCR refers to a “TCR that recognizes lipid antigen presented by a CD Id molecule.” It may include an alpha-TCR, a beta-TCR, or both. In some cases, the TCR utilized can belong to a broader group of “invariant TCR”, such as a MAIT cell TCR, GEM cell TCR, or gamma/delta TCR, resulting in HSC-engineered MAIT cells, GEM cells, or gamma/delta T cells, respectively.

In certain embodiments, a suicide gene is enzyme-based, meaning the gene product of the suicide gene is an enzyme and the suicide function depends on enzymatic activity. One or more suicide genes may be utilized in a single cell or clonal population. In some embodiments, the suicide gene encodes herpes simplex virus thymidine kinase (HSV-TK), purine nucleoside phosphorylase (PNP), cytosine deaminase (CD), carboxypetidase G2, cytochrome P450, linamarase, beta-lactamase, nitroreductase (NTR), carboxypeptidase A, or inducible caspase 9. Methods in the art for suicide gene usage may be employed, such as in U.S. Pat. No. 8,628,767, U.S. Patent Application Publication 20140369979, U.S. 20140242033, and U.S. 20040014191, all of which are incorporated by reference in their entirety. In further embodiments, a TK gene is a viral TK gene, i.e., a TK gene from a vims. In particular embodiments, the TK gene is a herpes simplex virus TK gene. In some embodiments, the suicide gene product is activated by a substrate. Thymidine kinase is a suicide gene product that is activated by ganciclovir, penciclovir, or a derivative thereof. In certain embodiments, the substrate activating the suicide gene product is labeled in order to be detected. In some instances, the substrate that may be labeled for imaging. In some embodiments, the suicide gene product may be encoded by the same or a different nucleic acid molecule encoding one or both of TCR-alpha or TCR-beta. In certain embodiments, the suicide gene is sr39TK or inducible caspase 9. In alternative embodiments, the cell does not express an exogenous suicide gene. In some embodiments, the engineered iNKT cell specifically binds to alpha-galactosylceramide (a-GC).

In additional embodiments, a cell is lacking or has reduced surface expression of at least one HLA-I or HLA-II molecule. In some embodiments, the lack of surface expression of HLA-I and/or HLA-P molecules is achieved by disrupting the genes encoding individual HLA-I/II molecules, or by disrupting the gene encoding B2M (beta 2 microglobulin) that is a common component of all HLA-I complex molecules, or by discrupting the genes encoding CIITA (the class II major histocompatibility complex transactivator) that is a critical transcription factor controlling the expression of all HLA-II genes. In specific embodiments, the cell lacks the surface expression of one or more HLA-I and/or HLA-II molecules, or expresses reduced levels of such molecules by (or by at least) 50, 60, 70, 80, 90, 100% (or any range derivable therein). In some embodiments, the HLA-I or HLA-II are not expressed in the iNKT cell because the cell was manipulated by gene editing.

In some embodiments, an iNKT cell comprises a recombinant vector or a nucleic acid sequence from a recombinant vector that was introduced into the cells. In certain embodiments the recombinant vector is or was a viral vector. In further embodiments, the viral vector is or was a lentivirus, a retrovirus, an adeno-associated virus (AAV), a herpesvirus, or adenovirus. It is understood that the nucleic acid of certain viral vectors integrate into the host genome sequence.

A “vector” or “construct” (sometimes referred to as gene delivery or gene transfer “vehicle”) refers to a macromolecule, complex of molecules, or viral particle, comprising a polynucleotide to be delivered to a host cell, either in vitro or in vivo. The polynucleotide can be a linear or a circular molecule. A “plasmid”, a common type of a vector, is an extra-chromosomal DNA molecule separate from the chromosomal DNA which is capable of replicating independently of the chromosomal DNA. In certain cases, it is circular and double-stranded.

A “gene,” “transgene”, “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “cell” is herein used in its broadest sense in the art and refers to a living body which is a structural unit of tissue of a multicellular organism, is surrounded by a membrane structure which isolates it from the outside, has the capability of self-replicating, and has genetic information and a mechanism for expressing it. Cells used herein may be naturally occurring cells or artificially modified cells (e.g., fusion cells, genetically modified cells, etc.).

INKT cells are a small population of alpha beta T lymphocytes highly conserved from mice to humans. iNKT cells have been suggested to play important roles in regulating many diseases, including cancer, infections, allergies, and autoimmunity. When stimulated, iNKT cells rapidly release a large amount of effector cytokines, e.g., like IFN-gamma and IL-4, both as a cell population and at the single-cell level. These cytokines then activate various immune effector cells, such as natural killer cells and dendritic cells (DCs) of the innate immune system, as well as CD4 helper and CD8 cytotoxic conventional alpha beta T cells of the adaptive immune system via activated DCs. Because of their unique activation mechanism, iNKT cells can attack multiple diseases independent of antigen, and MHC, restrictions, making them attractive universal therapeutic agents.

Previously, a series of iNKT cell-based clinical trials have been conducted, mainly targeting cancer. A recent trial reported encouraging anti-tumor immunity in patients with head and neck squamous cell carcinoma, attesting to the potential of iNKT cell-based immunotherapies. However, most clinical trials to date have yielded unsatisfactory results since they are based on the direct activation or ex vivo expansion of endogenous iNKT cells, thereby yielding only short-term, limited clinical benefits to a small number of patients. The low frequency and high variability of iNKT cells in humans (about 0.01-1% in blood), as well as the rapid depletion of these cells post-activation, are considered to be the major stumbling blocks limiting the success of these trials.

iNKT cells have been engineered from induced pluripotent stem (iPS) cells. See U.S. Pat. No. 8,945,922, incorporated by reference. iPS cells are produced by transducing a somatic cell with exogenous nuclear reprogramming factors, Oct4, Sox2, Klf4, and c-Myc, or the like. Unfortunately, since the transcription level of the exogenous nuclear reprogramming factors decreases with cell transition into the pluripotent state, the efficiency of stable iPS cell line production can decrease. Additionally, transcription of the exogenous nuclear reprogramming factors can resume in iPS cells and cause neoplastic development from cells derived from iPS cells since Oct4, Sox2, Klf4, and c-Myc are oncogenes that lead to oncogenesis.

Methods of the invention may be used to produce iNKT cells, for example, as discussed in U.S. Pub. No. US20170283481A1, and in World Application No. 2019241400, each of which are incorporated by reference.

As an example, in some embodiments, methods of the invention produce iNKT cells in which the iNKT TCR nucleic acid sequence is obtained from a subset of iNKT cells, such as the CD4/DN/CD8 subsets or the subsets that produce Th1, Th2, or Th17 cytokines, and includes double negative iNKT cells. In some embodiments, the iNKT TCR nucleic acid sequence is obtained from an iNKT cell from a donor who had or has a cancer such as melanoma, kidney cancer, lung cancer, prostate cancer, breast cancer, lymphoma, leukemia, a hematological malignancy, and the like. In some embodiments, the iNKT TCR nucleic acid molecule has a TCR alpha sequence from one iNKT cell and a TCR beta sequence from a different iNKT cell. In some embodiments, the iNKT cell from which the TCR alpha sequence is obtained and the iNKT cell from which the TCR beta sequence is obtained are from the same donor. In some embodiments, the donor of the iNKT cell from which the TCR alpha sequence is obtained is different from the donor of the iNKT cell from which the TCR beta sequence is obtained. In some embodiments, the TCR alpha sequence and/or the TCR beta sequence are codon optimized for expression. In some embodiments, the TCR alpha sequence and/or the TCR beta sequence are modified to encode a polypeptide having one or more amino acid substitutions, deletions, and/or truncations compared to the polypeptide encoded by the unmodified sequence. In some embodiments, the iNKT TCR nucleic acid molecule encodes a T cell receptor that recognizes alpha-galactosylceramide (alpha-GalCer) presented on CD1d. In some embodiments, the iNKT TCR nucleic acid molecule is contained in an expression vector. In some embodiments, the expression vector is a lentiviral expression vector. In some embodiments, the expression vector is a MIG vector in which the iNKT TCR nucleic acid molecule replaces the IRES-EGFP segment of the MIG vector. In some embodiments, the expression vector is phiNKT-EGFP.

The term “chimeric antigen receptor” or “CAR” refers to engineered receptors, which graft an arbitrary specificity onto an immune effector cell. These receptors are used to graft the specificity of a monoclonal antibody onto a T cell; with transfer of their coding sequence facilitated by retroviral or lentiviral vectors. The receptors are called chimeric because they are composed of parts from different sources. The most common form of these molecules are fusions of single-chain variable fragments (scFv) derived from monoclonal antibodies, fused to CD3-zeta transmembrane and endodomain; CD28 or 41BB intracellular domains, or combinations thereof. Such molecules result in the transmission of a signal in response to recognition by the scFv of its target. An example of such a construct is 14g2a-Zeta, which is a fusion of a scFv derived from hybridoma 14g2a (which recognizes disialoganglioside GD2). When T cells express this molecule (as an example achieved by oncoretroviral vector transduction), they recognize and kill target cells that express GD2 (e.g. neuroblastoma cells). To target malignant B cells, investigators have redirected the specificity of T cells using a chimeric immunoreceptor specific for the B-lineage molecule, CD19. The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows the scFv to orient in different directions to enable antigen binding. The transmembrane domain is a typical hydrophobic alpha helix usually derived from the original molecule of the signalling endodomain which protrudes into the cell and transmits the desired signal.

Preferably, the CAR is directed to a particular tumor antigen. Examples of tumor cell antigens to which a CAR may be directed include at least 5T4, 8H9, anbb integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FRcc, GD2, G250/CAIX, GD3, Glypican-3 (GPC3), Her2, IL-13Rcx2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-, BAGE family, CAGE family, GAGE family, MAGE family, SAGE family, XAGE family, NY-ESO-1/LAGE-1, PAME, SSX-2, Melan-A/MART-1, GP100/pmell7, TRP-1/-2, P. polypeptide, MC1R, Prostate-specific antigen, b-catenin, BRCA1/2, CML66, Fibronectin, MART-2, TGF\hat{ }RII, or VEGF receptors (e.g., VEGFR2), for example. The CAR may be a first, second, third, or more generation CAR. The CAR may be bispecific for any two nonidentical antigens, or it may be specific for more than two nonidentical antigens.

In some embodiments, a nucleic acid may comprise a nucleic acid sequence encoding an a-TCR and/or a b-TCR, as discussed herein. In certain embodiments, one nucleic acid encodes both the a-TCR and the b-TCR. In additional embodiments, a nucleic acid further comprises a nucleic acid sequence encoding a suicide gene product. In some embodiments, a nucleic acid molecule that is introduced into a selected CD34+ cell encodes the a-TCR, the b-TCR, and the suicide gene product. In other embodiments, a method also involves introducing into the selected CD34+ cells a nucleic acid encoding a suicide gene product, in which case a different nucleic acid molecule encodes the suicide gene product than a nucleic acid encoding at least one of the TCR genes.

Methods for preparing, making, manufacturing, and using engineered iNKT cells and iNKT cell populations are provided. Methods include 1, 2, 3, 4, 5, 6, 7, 8, 9, 10, 11, 12, 13, 14, 15 or more of the following steps in embodiments: obtaining hematopoietic cells; obtaining hematopoietic progenitor cells; obtaining progenitor cells capable of becoming one or more hematopoietic cells; obtaining progenitor cells capable of becoming iNKT cells; selecting cells from a population of mixed cells using one or more cell surface markers; selecting CD34+ cells from a population of cells; isolating CD34+ cells from a population of cells; separating CD34+ and CD34− cells from each other; selecting cells based on a cell surface marker other than or in addition to CD34; introducing into cells one or more nucleic acids encoding an iNKT T-cell receptor (TCR); infecting cells with a viral vector encoding an iNKT T-cell receptor (TCR); transfecting cells with one or more nucleic acids encoding an iNKT T-cell receptor (TCR); transfecting cells with an expression construct encoding an iNKT T-cell receptor (TCR); integrating an exogenous nucleic acid encoding an iNKT T-cell receptor (TCR) into the genome of a cell; introducing into cells one or more nucleic acids encoding a suicide gene product; infecting cells with a viral vector encoding a suicide gene product; transfecting cells with one or more nucleic acids encoding a suicide gene product; transfecting cells with an expression construct encoding a suicide gene product; integrating an exogenous nucleic acid encoding a suicide gene product into the genome of a cell; introducing into cells one or more nucleic acids encoding one or more polypeptides and/or nucleic acid molecules for gene editing; infecting cells with a viral vector encoding one or more polypeptides and/or nucleic acid molecules for gene editing; transfecting cells with one or more nucleic acids encoding one or more polypeptides and/or nucleic acid molecules for gene editing; transfecting cells with an expression construct encoding one or more polypeptides and/or nucleic acid molecules for gene editing; integrating an exogenous nucleic acid encoding one or more polypeptides and/or nucleic acid molecules for gene editing; editing the genome of a cell; editing the promoter region of a cell; editing the promoter and/or enhancer region for an iNKT TCR gene; eliminating the expression one or more genes; eliminating expression of one or more HLA-I/II genes in the isolated human CD34+ cells; transfecting into a cell one or more nucleic acids for gene editing; culturing isolated or selected cells; expanding isolated or selected cells; culturing cells selected for one or more cell surface markers; culturing isolated CD34+ cells expressing iNKT TCR; expanding isolated CD34+ cells; culturing cells under conditions to produce or expand iNKT cells; culturing cells in an artificial thymic organoid (ATO) system to produce iNKT cells; culturing cells in serum-free medium; culturing cells in an ATO system, wherein the ATO system comprises a 3D cell aggregate comprising a selected population of stromal cells that express a Notch ligand and a serum-free medium. It is specifically contemplated that one or more steps may be excluded in an embodiment.

Cells that may be used to create engineered iNKT cells are hematopoietic progenitor stem cells. Cells may be from peripheral blood mononuclear cells (PBMCs), bone marrow cells, fetal liver cells, embryonic stem cells, cord blood cells, or a combination thereof. The present disclosure encompasses “HSC-iNKT cells”, invariant natural killer T (iNKT) cells engineered from hematopoietic stem cells (HSCs) and/or hematopoietic progenitor cells (HPCs), and methods of making and using thereof. As used herein, “HSCs” is used to refer to HSCs, HPCs, or both HSCs and HPCs. “Hematopoietic stem and progenitor cells” or “hematopoietic precursor cells” refers to cells that are committed to a hematopoietic lineage but are capable of further hematopoietic differentiation and include hematopoietic stem cells, multipotential hematopoietic stem cells (hematoblasts), myeloid progenitors, megakaryocyte progenitors, erythrocyte progenitors, and lymphoid progenitors. “Hematopoietic stem cells (HSCs)” are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T-cells, B-cells, NK-cells). In this disclosure, HSCs refer to both “hematopoietic stem and progenitor cells” and “hematopoietic precursor cells”. The hematopoietic stem and progenitor cells may or may not express CD34. The hematopoietic stem cells may co-express CD 133 and be negative for CD38 expression, positive for CD90, negative for CD45RA, negative for lineage markers, or combinations thereof. Hematopoietic progenitor/precursor cells include CD34(+)/CD38(+) cells and CD34(+)/CD45RA(+)/lin(−)CD10+(common lymphoid progenitor cells), CD34(+)CD45RA(+)lin(−)CD10(−)CD62L(hi) (lymphoid primed multipotent progenitor cells), CD34(+)CD45RA(+)lin(−)CD10(−)CD123+(granulocyte-monocyte progenitor cells), CD34(+)CD45RA(−)lin(−)CD10(−)CD123+(common myeloid progenitor cells), or CD34(+)CD45RA(−)lin(−)CD10(−)CD123− (megakaryocyte-erythrocyte progenitor cells).

Certain methods involve culturing selected CD34+ cells in media prior to introducing one or more nucleic acids into the cells. Culturing the cells can include incubating the selected CD34+ cells with media comprising one or more growth factors. In some embodiments, one or more growth factors comprise c-kit ligand, flt-3 ligand, and/or human thrombopoietin (TPO). In further embodiments, the media includes c-kit ligand, flt-3 ligand, and TPO. In some embodiments, the concentration of the one or more growth factors is between about 5 ng/ml to about 500 ng/ml with respect to either each growth factor or the total of any and all of these particular growth factors. The concentration of a single growth factor or the combination of growth factors in media can be about, at least about, or at most about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500 (or any range derivable) ng/ml or mg/ml or more.

In some embodiments, cells are cultured in cell-free medium. In certain embodiments, the serum-free medium further comprises externally added ascorbic acid. In particular embodiments, methods involve adding ascorbic acid medium. In further embodiments, the serum-free medium further comprises 1, 2, 3, 4, 5, 6, 7, 8, 9, 10, 11, 12, 13, 14, 15, or all 16 (or a range derivable therein) of the following externally added components: FLT3 ligand (FLT3L), interleukin 7 (IL-7), stem cell factor (SCF), thrombopoietin (TPO), stem cell factor (SCF), IL-2, IL-4, IL-6, IL-15, IL-21, TNF-alpha, TGF-beta, interferon-gamma, interferon-lambda, TSLP, thymopentin, pleotrophin, or midkine. In additional embodiments, the serum-free medium comprises one or more vitamins. In some cases, the serum-free medium includes 1, 2, 3, 4, 5, 6, 7, 8, 9, 10, 11, or 12 of the following vitamins (or any range derivable therein): comprise biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or a salt thereof. In certain embodiments, medium comprises or comprise at least biotin, DL alpha tocopherol acetate, DL alpha-tocopherol, vitamin A, or combinations or salts thereof. In additional embodiments, serum-free medium comprises one or more proteins. In some embodiments, serum-free medium comprises 1, 2, 3, 4, 5, 6 or more (or any range derivable therein) of the following proteins: albumin or bovine serum albumin (BSA), a fraction of BSA, catalase, insulin, transferrin, superoxide dismutase, or combinations thereof. In other embodiments, serum-free medium comprises 1, 2, 3, 4, 5, 7, 8, 9, 10, or 11 of the following compounds: corticosterone, D-Galactose, ethanolamine, glutathione, L-camitine, linoleic acid, linolenic acid, progesterone, putrescine, sodium selenite, or triodo-I-thyronine, or combinations thereof. In further embodiments, serum-free medium comprises a B-27 supplement, xeno-free B-27 supplement, GS21™ supplement, or combinations thereof. In additional embodiments, serum-free medium comprises or further comprises amino acids, monosaccharides, and/or inorganic ions. In some aspects, serum-free medium comprises 1, 2, 3, 4, 5, 6, 7, 8, 9, 10, 11, 12, or 13 of the following amino acids: arginine, cysteine, isoleucine, leucine, lysine, methionine, glutamine, phenylalanine, threonine, tryptophan, histidine, tyrosine, or valine, or combinations thereof. In other aspects, serum-free medium comprises 1, 2, 3, 4, 5, or 6 of the following inorganic ions: sodium, potassium, calcium, magnesium, nitrogen, or phosphorus, or combinations or salts thereof. In additional aspects, serum-free medium comprises 1, 2, 3, 4, 5, 6 or 7 of the following elements: molybdenum, vanadium, iron, zinc, selenium, copper, or manganese, or combinations thereof.

In some methods, cells are cultured in an artificial thymic organoid (ATO) system. The ATO system involves a three-dimensional (3D) cell aggregate, which is an aggregate of cells. In certain embodiments, the 3D cell aggregate comprises a selected population of stromal cells that express a Notch ligand. In some embodiments, a 3D cell aggregate is created by mixing CD34+ transduced cells with the selected population of stromal cells on a physical matrix or scaffold. In further embodiments, methods comprise centrifuging the CD34+ transduced cells and stromal cells to form a cell pellet that is placed on the physical matrix or scaffold. In certain embodiments, stromal cells express a Notch ligand that is an intact, partial, or modified DLL1, DLL4, JAG1, JAG2, or a combination thereof. In further embodiments, the Notch ligand is a human Notch ligand. In other embodiments, the Notch ligand is human DLL1.

Cells may be used immediately or they may be stored for future use. In certain embodiments, cells that are used to create iNKT cells are frozen, while produced iNKT cells may be frozen in some embodiments. In some aspects, cells are in a solution comprising dextrose, one or more electrolytes, albumin, dextran, and DMSO. In other embodiments, cells are in a solution that is sterile, nonpyrogenic, and isotonic. In some embodiments, the engineered iNKT cell is derived from a hematopoietic stem cell. In some embodiments, the engineered iNKT cell is derived from a G-CSF mobilized CD34+ cells. In some embodiments, the cell is derived from a cell from a human patient that doesn't have cancer. In some embodiments, the cell doesn't express an endogenous TCR.

Engineered iNKT cells may be used to treat a patient. In some embodiments, methods include introducing one or more additional nucleic acids into the cell population, which may or may not have been previously frozen and thawed. This use provides one of the advantages of creating an off-the-shelf iNKT cell. In particular embodiments, the one or more additional nucleic acids encode one or more therapeutic gene products. Examples of therapeutic gene products include at least the following: 1. Antigen recognition molecules, e.g. CAR (chimeric antigen receptor) and/or TCR (T cell receptor); 2. Co-stimulatory molecules, e.g. CD28, 4-1BB, 4-1BBL, CD40, CD40L, ICOS; and/or 3. Cytokines, e.g. IL-1cc, IL-Ib, IL-2, IL-4, IL-6, IL-7, IL-9, IL-15, IL-12, IL-17, IL-21, IL-23, IFN-g, TNF-α, TGF-b, G-CSF, GM-CSF; 4. Transcription factors, e.g. T-bet, GATA-3, RORyt, FOXP3, and Bcl-6. Therapeutic antibodies are included, as are chimeric antigen receptors, single chain antibodies, monobodies, humanized, antibodies, bi-specific antibodies, single chain FV antibodies or combinations thereof.

In some embodiments, the present invention provides kits comprising one or more engineered cells or compositions according to the present invention packaged together with a drug delivery device, e.g., a syringe, for delivering the engineered cells or compositions to a subject. In some embodiments, the present invention provides kits comprising one or more engineered cells or compositions according to the present invention packaged together with one or more reagents for culturing and/or storing the engineered cells. In some embodiments, the present invention provides kits comprising one or more engineered cells or compositions according to the present invention packaged together with one or more agents that activate cells, e.g., iNKT cells comprising a CAR and at least one additional transgene. In some embodiments, the present invention provides kits comprising one or more engineered cells or compositions according to the present invention packaged together with OP9-DL1 stromal cells and/or MS5-DL4 stromal cells. In some embodiments, the present invention provides kits comprising one or more engineered cells or compositions according to the present invention packaged together with antigen-presenting cells or CD1d-expressing artificial antigen-presenting cells.

Methods of the invention provides methods of manufacturing engineered cells for treating any number of conditions and/or diseases. In some embodiments, the present invention provides a method of treating a subject, which comprises administering to the subject one or more engineered cells according to the present invention, one or more engineered cells made according to a method of the present invention, or one or more compositions according to the present invention. In some embodiments, the subject is an animal such as a mouse or a test animal. In some embodiments, the subject is a human. In some embodiments, the subject has a cancer, a bacterial infection, a viral infection, an allergy, or an autoimmune disease. In some embodiments, the cancer is melanoma, kidney cancer, lung cancer, prostate cancer, breast cancer, lymphoma, leukemia, or a hematological malignancy. In some embodiments, the subject has tuberculosis, HIV, or hepatitis. In some embodiments, the subject has asthma or eczema. In some embodiments, the subject has Type I diabetes, multiple sclerosis, or arthritis. In some embodiments, the subject is administered a therapeutically effective amount of the one or more engineered cells. In some embodiments, the therapeutically effective amount of the one or more engineered cells is about 10×107 to about 10×109 cells per kg body weight of the subject being treated. In some embodiments, the method further comprises administering an agent that activates iNKT cells, e.g., α-GalCer or salts or esters thereof, α-GalCer-presenting dendritic cells or artificial APCs, before, during, and/or after administration of the one or more engineered cells.

In certain aspects, this disclosure provides systems and methods for producing T cells with enhanced anti-tumor phenotypes. In particular, this disclosure provides methods of making T cells from stem cells (e.g., HSCs) engineered with multiple transgenes including a T cell receptor (TCR), a chimeric antigen receptor (CAR), and at least one additional transgene. By starting with HSCs, systems and methods of the invention take advantage of self-regeneration and cellular differentiation capabilities of stem cells for the manufacture of T cells with improved anti-tumor phenotypes. In particular, this disclosure provides for introduction of nucleic acids, into CD34+ stem cells, that encode a TCR, a CAR, and at least one an additional transgene. The combined expression of TCRs and CARs is useful for providing T cells with specific cancer cell targeting properties. Moreover, endowed with at least one additional transgene, the T cells produced by methods of the invention are armed with cargo (e.g., cytokines) that, when in contact with the target cancer cell, is useful to treat the cancer.

For example, in preferred embodiments, methods of the invention involve introducing nucleic acids into HSCs via lentiviral transduction. Introduction of the one or more nucleic acids provides for HSCs that express at least one TCR, CAR, and an additional transgene. Incorporation of the additional transgene is useful for providing therapeutic T cells with improved functional properties, such as, improved cell expansion, persistence, safety, and/or antitumor activities.

The one or more additional transgenes may include any one or more of a cytokine, a checkpoint inhibitor, an inhibitor of transforming growth factor beta signaling, an inhibitor of cytokine release syndrome, or an inhibitor of neurotoxicity. For example, transgenes may be provided that encode one or more of IL-2, IL-7, IL-15, IL-12, IL-18, or IL-21.

Accordingly, in some instances, methods of the invention provide for the manufacture of CAR T cells with improved expansion and persistence capabilities by, for example, introducing nucleic acids encoding one or more of IL-2, IL-7, IL-1-15. In some instances, methods may provide CAR T cells with increased IFN-g production and thus improved T cell potency by, for example, introduction of transgenes encoding one or more of IL-12, IL-18. In other instances, methods of the invention are useful for enhancing naïve T cell production by introducing transgenes including IL-21. In some instances, methods described herein provide for the production of CAR T cells with improved safety properties by, for example, introducing inhibitors of IL-6, GM-CSF, or other mediators of cytokine release syndrome and neurotoxicity. Methods may provide for CAR T cells with improved efficacy by providing payloads useful for combating tumor microenvironment, e.g., via inhibitors of TGF-B, checkpoints.

Certain methods of the invention involve a single in vitro activation step. In some instances, the T cells are briefly activated with reagents, e.g., for 1-3 days and following this, activation reagents are often removed from the media so as to not continuously stimulate cells and thus exhaust the cell. Following activation, an activated T cell population may expand rapidly, e.g., double in number every 24 hours. Some reagents may be added to facilitate the expansion. In some embodiments, the method involves a culture media may be supplemented with one or more of IL-7/15, IL-2, IL-2+21, IL-12, or IL-18.

In some instances, the single activation step involves PMBC-based T cell activation. Accordingly, the activation step may involve introducing aGC-loaded PBMCs, soluble anti-CD3/28 positive PBMCs, and soluble anti-CD2/3/28 positive PBMCs to the T cell. In some instances, the activation step involves an antigen presenting cell (aAPC) based T cell activation step. Accordingly, the activation step can involve introducing the T cell to aAPC. The aAPC may be an engineered K562 cell expressing CD80-CD83-CD137L-CAR-antigen. The aAPC may be an aAPC+CD1d, and/or aAPC+CD1d+/−aGC. In other instances, the activation step comprises a feeder free-based T cell activation step. The feeder free based T cell activation step can involve introducing, to the T cell, soluble antibodies including anti-CD3, anti-CD28, anti-CD2/3/28, CD3/28. In some embodiments, the T cell activation step involves culturing the T cell in the presence of different cytokines added to activations expansion culture media IL-7/15, IL-2, IL-2+21, IL-12, IL-18.

The following examples provide useful exemplary protocols for manufacture of T cells (e.g., iNKTs) from CD34+ cells as provided by methods of the invention. For further examples and discussion, see WO2019241400A1, which is incorporated by reference.

### Example 1: CD34+ HSPCs Cell Culture and Lentiviral Transduction

### Day-2: Pre-stimulation

1. Coat appropriate number of wells (recommended to seed ˜15×10³ cells/well or 6 wells for a 0.1×10⁶ aliquot) of a 24-well non-tissue culture treated plate with 0.5 mL/well of 20 ug/ml retronectin (RN) diluted in PBS. 1 mg/mL RN stock aliquots may be stored at −20 degrees Celsius in 60 microliter aliquots.
2. Incubate at room temperature (RT) for 2 hours (h).
3. Aspirate RN and replace with 0.5 ml/well of 2 percent BSA diluted in PBS. 30 percent BSA aliquots are stored at −20° Celsius.
4. Incubate for 30 min at RT.
5. Aspirate and replace with 0.5 ml/well of PBS.
6. Thaw a 0.1×10⁶ aliquot of CD34+ cells into Stem Cell Media following standard procedures and spin at 300 g for 10 min.
7. Aspirate supernatant, resuspend in Stem Cell Media, and count using hemocytometer, record cell count. In some instances, applicant's found cell counts for a 0.1×10⁶ aliquot were too low to be reliable.
8. Dilute CD34+ cells with Stem Cell Media to 0.05×10⁶ cells/ml.
9. Aspirate PBS from RN-coated wells and seed 300 ul cells/well (˜15×10³ cells/well).
10. Incubate at 37 degrees Celsius, 5 percent CO2 for 12-18 h.

### Day-1: Transduction

1. Thaw concentrated lentiviral vector (LVV) and pipet gently to mix (do not vortex and do not refreeze).
2. Prepare transduction tube:
a. Transfer appropriate volume of LVV to 1.5 ml tube by calculating volume sufficient for a MOI of 100-200.
b. Add appropriate volume of PGE2 to same tube to achieve a final concentration of 10 nM of total culture volume.
c. Add appropriate volume of poloxamer to same tube to achieve a final concentration of 1 ug/ul of total culture volume.
3. Add contents of transduction tube to appropriate culture well and rock plate gently to mix
4. Incubate cells at 37 degrees Celsius, 5 percent CO2 for 24 h.

### Day 0 Harvest

1. Collect cells by pipetting gently to remove them from the plate and transfer to conical tube.
2. Wash wells with equal volume of cold X-VIVO-15 to remove any cells still adhering to plate and transfer to conical tube.
3. Check under microscope and perform additional washes with cold X-VIVO-15 as necessary to collect all cells from plate.
4. Spin at 300 g for 10 minutes and aspirate supernatant.
5. Proceed to Stage 2 (i.e., Example 2).

### Example 2: Generation of iNKT-CAR Cells: Differentiation

Days 0-14: Differentiation (Duration: 2 weeks)
1. Coat appropriate number of wells (recommended to seed 1,000-2,000 cells/well or 12 wells/15,000 cells) of 12-well non-tissue culture-treated plates with 1 ml/well with a lymphoid differentiation coating material (LDCM), such as, the lymphoid differentiation material provided under the trade name StemSpan by STEMCELL, diluted 1:200 in PBS. Incubate 12-18 h at 4 degrees Celsius.
2. Aspirate LDCM and add 2 ml/well of PBS
3. Resuspend transduced CD34+ cells collected in Stage 1 (i.e., Example 1) in a lymphoid progenitor expansion medium (LPEM), such as, the lymphoid progenitor expansion medium sold under the trade name StemSpan by STEMCELL.
4. Adjust cell density to 1-2×10³ cells/ml with LPEM
5. Aspirate PBS from LDCM-coated plates
6. Seed 0.75 ml cells/well into a LCDM-coated plate
7. Incubate cells at 37 degrees Celsius, 5 percent CO2
8. On day 3, add 0.25 ml/well of fresh LPEM and continue culture
9. On days 7 and 11, carefully remove <0.5 ml/well without disturbing cells and replenish with 0.5 ml/well of fresh LPEM
10. Continue culture to day 14
11. Proceed to Stage 3 (i.e., Example 3)

### Example 3: Generation of iNKT-CAR Cells: Maturation

### Day 14-21+: Maturation (Duration: 1-2 Weeks)

1. Coat appropriate number of wells (recommended to seed 50-100×10³/well) of 6-well non-tissue culture-treated plates with 2 ml/well of LDCM diluted 1:200 in PBS. Incubate 12-18 h at 4 degrees Celsius if preparing a day prior to seeding or at 37 degrees Celsius for 2 h if preparing the day of seeding.
2. Aspirate LDCM and add 4 mL/well of PBS
3. On day 14, harvest and count cells using a cell viability counter, such as, the cell viability counter provided under the trade name Vi-Cell XR by Beckman Coulter.
   a. Collect cells by pipetting gently to remove them from the plate and transfer to conical tube.
   b. Wash wells with 1 ml/4 wells of cold SFEM II to remove any cells still adhering to plate and transfer to conical tube
   c. Check under microscope and perform additional washes with cold SFEM II as necessary to collect cells from plate.
4. Pull an aliquot of 0.2×10⁶ cells and seed into a 96-well V-bottom plate for flow staining.
5. Pull an appropriate volume of cells to seed for Stage 3 and pellet at 300 g for 10 min.
6. Aspirate supernatant.
7. Resuspend cells in T cell progenitor maturation medium (TPMM), such as, the T cell progenitor maturation medium provided under the trade name StemSpan by STEMCELL, at 2.5-5×10⁴ cells/ml.
8. Aspirate PBS from LDCM-coated plates.
9. Seed 2 ml cells/well into LDCM-coated plate.
10. Incubate cells at 37 degrees Celsius, 5 percent CO2
11. Pellet remaining cells at 300 g for 10 min.
12. Aspirate and resuspend remaining cells in appropriate volume of cryopreservation solution, such as, the cryopreservation solution sold under the trade name CryoStor CS10 by STEMCELL, to achieve 2-5×10⁶ cells/mL.
13. Aliquot 1 ml/cryovial and freeze appropriately (e.g., in a negative 80 degrees Celsius freezer); move to liquid nitrogen storage within 24 hours of freezing.
14. On day 17, add 2 ml/well of fresh TPMM and continue culture.
15. On day 21, take sample for flow cytometry by pipetting 100 ul from cells in center of well and seed into a 96-well V-bottom plate for flow staining.
16. Take another 100 ul from cells in center of well and count using cell viability counter.
17. If TCR expression is >50%, CD4/CD8 double positive expression is >20%, and cell size has decreased (indicative of development from HSC to T cells), cells may cryopreserved and provided for allogenic therapy, or optionally, expanded as provided by Stage 4. If these parameters are not met, continue culture with necessary feeding and splitting. Remove <2 ml/well and replenish with 2 ml/well of fresh TPMM every 3-4 days and splitting cultures if they approach confluence. Perform this check again on day 24/25 and day 28 until above criteria are met.
   Stage 4: Generation of iNKT-CAR Cells: Optional Expansion:
Day 21-28 (assuming criteria met at day 21, adjust accordingly): Stimulation (Duration: 1 week)
1. On day 21, harvest and count cells using cell viability counter (record counts in associated excel sheet)
   a. Collect cells by pipetting gently to remove them from the plate and transfer to conical tube
   b. Wash wells with 2 ml per 4 wells of cold cell expansion medium (EM), such as, the cell expansion medium provided under the trade name OpTmizer by ThermoFisher, to remove any cells still adhering to plate and transfer to conical tube
   c. Check under microscope and perform additional washes with cold EM as necessary to collect cells from plate.
   d. Pull an aliquot of 0.2×10⁶ cells and seed into a 96-well V-bottom plate for flow staining.
   e. Pull an appropriate volume of cells to seed for Stage 4 and pellet at 300 g for 10 min.
   f. Aspirate supernatant.
   g. Resuspend cells at 2×10⁶ cells per ml in EM with IL-7 and IL-15 at 10 ng/ml
2. Follow subset of following instructions depending on desired method(s) of stimulation. iNKT-CAR cell final concentration is fixed between conditions at 1×10⁶ ml
   a. No stimulation
      i. Dilute cells to 1×10⁶ cells per ml with EM media with IL-7 and IL-15 at 10 ng/ml
      ii. Seed cells and incubate at 37 degrees Celsius, 5 percent CO2
   b. Coated CD3/Soluble CD28
      i. Coat plates with 1.23ug/ml CD3 for 2 hours at 37° C. and wash with PBS before using.
      ii. Dilute cells to 1×10⁶ cells per ml with EM with IL-7 and IL-15 at 10 ng/ml.
      iii. Add soluble CD28 to cell suspension at 1 ug/ml.
      iv. Seed cells and incubate at 37 degrees Celsius, 5 percent CO2.
   c. Soluble CD3/Soluble CD28 with PBMCs
      i. Thaw human peripheral blood mononuclear cells (PBMCs) and irradiate at 6,000 rads.
      ii. Resuspend PBMCs EM with IL-7 and IL-15 at 10 ng/ml.
      iii. Combine iNKT-CAR cells with PBMCs at a 1:2-3 (iNKT-CAR:PBMC) ratio so that the iNKT-CAR cell final concentration is 1×10⁶ cells per ml.
      iv. Add soluble CD3 and soluble CD28 to cell suspension at 1 ug/ml.
      v. Seed cells and incubate at 37 degrees Celsius, 5 percent CO2.
   d. Provide a CD3/CD28 T Cell Activator, such as, the activator sold under the trade name ImmunoCult Human CD3/CD28 T Cell Activator by StemCell Technologies.
      i. Dilute cells to 1×10⁶ cells per ml with EM media with IL-7 and IL-15 at 10 ng/ml.
      ii. Add CD3/CD28 T Cell Activator to cell suspension at 25ul/ml.
      iii. Seed cells and incubate at 37 degrees Celsius, 5 percent CO2.
   e. Provide CD3/CD28/CD2 T Cell Activator, such as, the activator sold under the trade name ImmunoCult Human CD3/CD28/CD2 T Cell Activator by StemCell Technologies.
      i. Dilute cells to 1×10⁶ cells per ml with EM with IL-7 and IL-15 at 10 ng/ml.
      ii. Add CD3/CD28/CD2 T Cell Activator to cell suspension at 25ul/ml.
      iii. Seed cells and incubate at 37 degrees Celsius, 5 percent CO2.
   f. aGC-loaded PBMCs
      i. Thaw human peripheral blood mononuclear cells (PBMCs).
      ii. Resuspend at 10×10⁶ cells per ml in EM with 2 ug/ml aGC and incubate at 37 degrees Celsius, 5 percent CO2 for 1 h.
      iii. Collected aGC-loaded PBMCs and irradiate at 6,000 rads.
      iv. Perform at least two washes of cells with EM to remove unbound aGC
      v. Resuspend PBMCs in EM with IL-7 and IL-15 at 10 ng/ml.
      vi. Combine iNKT-CAR cells with PBMCs at a 1:2-3 (iNKT-CAR:PBMC) ratio so that the iNKT-CAR cell final concentration is 1×10⁶ cells per ml.
      vii. Seed cells and incubate at 37 degrees Celsius, 5 percent CO2.
   g. K562-CD80-CD83-CD137L-A2ESO artificial antigen presenting cells (aAPC).
      i. Collect aAPCs from in culture and irradiate at 10,000 rads.
      ii. Combine iNKT-CAR cells with aAPCs at a 4:1 (iNKT-CAR:aAPC) ratio so that the iNKT-CAR cell final concentration is 1×10⁶ cells per ml.
      iii. Seed cells and incubate at 37 degrees Celsius, 5 percent CO2.
   h. K562-CD80-CD83-CD137L-A2ESO-CD1d artificial antigen presenting cells (aAPC-CD1d).
      i. Collect aAPCs from in culture and irradiate at 10,000 rads.
      ii. Combine iNKT-CAR cells with aAPCs at a 4:1 (iNKT-CAR:aAPC) ratio so that the iNKT-CAR cell final concentration is 1×10⁶ cells per ml.
      iii. Seed cells and incubate at 37 degrees Celsius, 5 percent CO2.
   i. aGC-loaded K562-CD80-CD83-CD137L-A2ESO-CD1d artificial antigen presenting cells (aAPC-CD1d).
      i. Collect aAPCs from in culture.
      ii. Resuspend at 10×10⁶ cells per ml in EM with 2ug/ml aGC (see α-GalCer Prep SOP) and incubate at 37° C., 5% CO2 for 1 h.
      iii. Collected aGC-loaded aAPCs and irradiate at 10,000 rads.
      iv. Combine iNKT-CAR cells with aAPCs at a 4:1 (iNKT-CAR:aAPC) ratio so that the iNKT-CAR cell final concentration is 1×10⁶ cells per ml.
      v. Seed cells and incubate at 37 degrees Celsius, 5 percent CO2.
3. On day 24 count cells using cell counter (record counts in associated excel sheet) and dilute cells to 1×10⁶cells per ml using EM with IL-7 and IL-15 at 10 ng/ml, transfer culture vessels as necessary. If utilizing PBMCs or aAPCs the cell counts taken at this timepoint may be harder to interpret, in this case carefully remove half of the culture media without disturbing the cells and add an equivalent volume of fresh EM with IL-7 and IL-15 at 10 ng/ml. If conditions without PBMCs or aAPCs do not require dilution also perform this half-media change.
4. On day 26 count cells using cell viability counter (record counts in associated excel sheet) and dilute cells to 1×10⁶cells per ml using EM with IL-7 and IL-15 at 10 ng/ml, transfer culture vessels as necessary.
5. On day 28 harvest cells and count using Vi-Cell (record counts in associated excel sheet)
   a. Pull an aliquot of 0.2×10⁶ cells and seed into a 96-well V-bottom plate for flow staining.
   b. Pull aliquots of 0.2×10⁶ cells and seed into a 96-well V-bottom plate for additional flow staining characterization.
   c. Pull aliquot of 1×10⁶ cells, dilute to 1×10⁶ cells per ml using EM with IL-7 and IL-15 at 1Ong/ml, and seed for longitudinal tracking (optional step, prioritize after cell freezing is completed).
   d. Pellet remaining cells at 300 g for 10 min and aspirate supernatant.
   e. Resuspend in appropriate volume of cryopreservation media to achieve 25-50×10⁶ cells per mL.
   f. Aliquot 1 ml/cryovial and freeze appropriately, move to liquid nitrogen storage within 24 hours of freezing.

### INCORPORATION BY REFERENCE

References and citations to other documents, such as patents, patent applications, patent publications, journals, books, papers, web contents, have been made throughout this disclosure.

All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of producing an engineered invariant natural killer T cell, the method comprising:
introducing into a hematopoietic stem cell (HSC) one or more nucleic acids encoding a T cell receptor (TCR), a chimeric antigen receptor (CAR), and at least one additional transgene; and
transforming the HSC into an invariant natural killer T (iNKT) cell that expresses the TCR and CAR and comprises the transgene.

2. The method of claim 1, wherein the one or more nucleic acids are provided by a single vector.

3. The method of claim 2, wherein the single vector comprises a lentiviral vector.

4. The method of claim 1, wherein introducing the one or more nucleic acids involves incorporating at least two distinct nucleic acid molecules into the HSC via a plurality of vectors.

5. The method of claim 4, wherein the at least two district nucleic acid molecules comprise lentiviral vectors.

6. The method of claim 4, wherein the at least two distinct nucleic acid molecules are introduced into the HSC sequentially or simultaneously.

7. The method of claim 1, wherein the HSC is derived from a progenitor cell.

8. The method of claim 7, wherein the progenitor cell is a pluripotent stem cell.

9. The method of claim 1, wherein the one or more nucleic acids further encode a sequence to induce ribosomal skipping.

10. The method of claim 9, wherein the sequence encodes a 2A sequence.

11. The method of claim 1, wherein the iNKT cell is an alpha/beta iNKT cell, or gamma/delta iNKT cell.

12. The method of claim 1, wherein the one or more additional transgenes comprise at least one of a cytokine, a checkpoint inhibitor, an inhibitor of transforming growth factor beta signaling, an inhibitor of cytokine release syndrome, or an inhibitor of neurotoxicity.

13. The method of claim 12, wherein the cytokine comprises one of IL-2, IL-7, IL-15, IL-12, IL-18, or IL-21.

14. The method of claim 1, wherein the CAR comprises a single domain antibody.

15. The method of claim 1, wherein the CAR comprises a variable region of a heavy-chain antibody.
